(19) United States
(12) Patent Application Publication
(10) Pub. No.: US 2020/0407673 A1
(43) Pub. Date: Dec. 31, 2020

(54) DEVICE, KIT AND METHOD FOR THREE-DIMENSIONAL CELL CULTURE

(71) Applicant: RIGENERAND S.R.L., Medolla (IT)

(72) Inventors: Massimo DOMINICI, Ferrara (IT); Olivia CANDINI, Crevalcore (IT); Matteo BROGLI, Ferrara (IT); Giorgio MARI, Mirandola (IT)

(73) Assignee: RIGENERAND S.R.L., Medolla (IT)

(21) Appl. No.: 16/962,152

(22) PCT Filed: Jan. 16, 2019
(86) PCT No.: PCT/IT2019/050005

§ 371 Date: Jul. 14, 2020

(30) Foreign Application Priority Data
Jan. 16, 2018 (IT) 102018000001123

(51) Int. Cl. C12M 1/12; C12M 1/00

(52) U.S. Cl. CPC C12M 25/14; C12M 29/10; C12M 29/04

(57) ABSTRACT

Three-dimensional cell culture device comprising: a container body of cells to be cultivated formed by a first semi-portion and a second semi-portion facing each other and attached together with attachment means; a culture compartment which is defined between the first semi-portion and the second semi-portion; a three-dimensional substrate for the engraftment and/or support of the cells to be cultivated which is located in the culture compartment; an inlet of a transport solution of cells to be cultivated, and an outlet that connects the culture compartment with the outside, to discharge the transport solution; at least one of said first semi-portion and second semi-portion comprises oxygenation means of the cells to be cultivated.

### FIELD OF THE INVENTION

The invention concerns a device, a kit and a method for three-dimensional cell culture, generally usable to cultivate cells in an extra-body environment and to verify the action of active principles on cultured cells inside the device.

### BACKGROUND OF THE INVENTION

Devices to cultivate cells are known, which consist of a container inside which a three-dimensional support or substrate is disposed on which the cells to be cultivated can engraft.

The container is shaped substantially like a box which can be parallelepiped or cylindrical and which comprises an inlet and an outlet to introduce a flow of a fluid in which the cells to be cultivated are transported and to discharge the fluid after it has released the cells on the three-dimensional support.

The box has an airtight seal element to guarantee insulation from the outside.

A device of this type is known from the American U.S. Pat. No. 5,843,766 which discloses an apparatus to cultivate and package cultivations of three-dimensional organic tissues.

Typically, the device comprises a base box-like body equipped with a lid and in which a culture chamber is defined and in which cells are cultivated to obtain three-dimensional organic tissues, such as for example skin flaps, which can be stored in a frozen environment and transported to the recipient in the same container, keeping them in an aseptic environment.

The container comprises a three-dimensional substrate which is located inside the culture chamber defined in the base box-like body, to promote the growth of the cells and, therefore, of the three-dimensional skin flap to be created.

As we said, the container is equipped with two doors which put the culture chamber in communication with the outside, that is, an inlet door for a fluid which transports the cells to be cultivated and an outlet door for the fluid after it has released the cells on the substrate.

To guarantee aseptic conditions inside the culture chamber, sealing gaskets are provided at the conjunction points of the components of the container, in particular between the base body and the closing lid.

The inside of the culture chamber is equipped with deflectors and/or walls to create a specific path of the fluid flow, so that the latter passes uniformly over the three-dimensional substrate and uniformly distributes thereon the cells to be cultivated.

The inside of the culture chamber is also equipped with raised pins to support and clamp the substrate, so as to both prevent it from accidentally moving during cultivation, and also so that it remains positioned equidistant from the walls of the box-like body and the lid.

The latter allows access into the culture chamber to remove the skin flaps obtained.

The state of the art has some disadvantages.

A first disadvantage is that the device must be equipped with specific deflectors and/or walls inside the culture chamber to make a shaped path in such a way as to divert the flow of the incoming fluid in predetermined directions.

Moreover, raised pins must also be provided to support and clamp the substrate in its correct position of use.

Typically, this requirement makes the overall structure of the culture device complicated.

A second disadvantage is that the substrate must be conformed so as to be able to support the skin flaps obtained with the culture, without the latter being damaged due to their very delicate structure.

Moreover, in order to prevent part of the epidermis to be obtained from being generated in unsuitable zones, it is necessary to provide that both the box-like body and the lid are made in such a way as to inhibit cell growth on them.

A technique is also known for evaluating the efficacy of drugs on healthy or pathology-affected cells, in particular tumor pathologies, using in vitro assays or in vivo assays.

In the case of in vitro assays, cell cultures known as mono-layer or also two-dimensional cultures are used.

In the case of in vivo assays, cell transplants are used which, if in this specific case are human tumor cells or normal human primary cells, occur in immuno-compromised xeno-transplanted mice.

These known techniques are currently the only ones available to evaluate the pharmacological or biological activity and safety of a therapeutic treatment, such as an anti-tumor treatment for example.

However, this known evaluation technique has several disadvantages.

A first disadvantage is that the mono-layer cell cultures are physiologically very different from the three-dimensional tissues that give origin to the cells themselves, such as tumorous tissues, which is why the anti-tumor drugs have shown a significantly different efficacy and power if evaluated on two-dimensional or three-dimensional cell cultures.

The reason for this diversity is determined by the different cell growth that is obtained in vitro on mono-layer cultures, compared to cell growth in three-dimensional cultures, due to some critical factors.

A first critical factor is mechanical, since, in mono-layer cultures, the cells are subjected to a condition of greater rigidity than the three-dimensional cultures that more accurately reflect the mechanical conditions between the forces exerted on the in vivo cells and, therefore, the conditions that are closest to reality.

A second critical factor of in vitro cultures is biochemical, since access to nutrient substances, that is, oxygen, ions, gradients and drugs, is critical for in vivo tissues and differs considerably in vitro due to the different disposition of the cells in mono-layer cultures compared to three-dimensional cultures.

A third critical factor is environmental, since the physiological interactions between cell and cell and their spatial conformation are highly compromised in mono-layer cultures.

All the critical factors indicated above can significantly influence the intracellular mechanisms of response to external stimuli, altering the gene and antigenic expression, and impacting on the conformation of the cell structure and on their phenotypic and differentiating state.

It is therefore desirable to be able to get as close as possible to the growth conditions of in vivo cells, simulating their natural microenvironment, in the case of a tumorous microenvironment, in such a way as to increase the predictive response capacity of an active principle or a therapeutic treatment.

It should also be considered that the evaluation of the efficacy of a pharmacological treatment in the in vivo animal models differs considerably from the in vitro assay also in terms of the number of tumor cells subjected to treatment.

In addition, because in vitro assays are miniaturized for convenience, they involve a significantly smaller and less representative number of cells than the number of cells that make up an in vivo tissue mass, in many cases compromising the predictive response to treatment, generating false positive feedback or false negative feedback, and thus negatively impacting the specificity and sensitivity of the test.

### PRESENTATION OF THE INVENTION

Purpose of the invention is to overcome the disadvantages of the state of the art. Another purpose of the invention is to perfect a device and a method for three-dimensional cell culture that allow to make cell cultures outside a living being that are as similar as possible to the life conditions of the cells in the original tissues in vivo.

Another purpose of the invention is to perfect a device and a method for three-dimensional cell culture which allow to give a highly reliable prediction on the safety or efficacy of an active principle and a therapeutic treatment, prior to their application on a living being.

Another purpose of the invention is to perfect a device and a method for three-dimensional cell culture that is easy to apply and handle, maintaining a high level of safety for the healthcare workers that use it and for the cells contained in the device.

According to one aspect of the invention a device for three-dimensional cell culture is provided, in accordance with the characteristics of claim 1.

According to another aspect of the invention, a kit for three-dimensional cell culture as in claim 9 is provided.

According to another aspect of the invention, a method for three-dimensional cell culture is provided, in accordance with the characteristics of claim 10.

Other aspects of the invention are indicated in the independent claims.

The invention allows to obtain the following advantages:
to obtain three-dimensional cell cultures in conditions very similar to natural ones;
to predict with high reliability the safety or efficacy of drugs and/or therapeutic treatments on healthy or diseased cells, before they are used on a living being.

### BRIEF DESCRIPTION OF THE DRAWINGS

Other characteristics and advantages of the invention will become more apparent from the detailed description of preferred but non-exclusive embodiments, of a device for three-dimensional cell culture, shown by way of a non-restrictive example with reference to the attached drawings wherein:

FIG. 1 is a schematic, perspective view of a device for three-dimensional cell-culture according to the invention;

FIG. 2 is a view of a first semi-portion that forms the device in FIG. 1;

FIG. 3 is a view of a first semi-portion that forms the device in FIG. 1;

FIG. 4 is a schematic view in longitudinal section of the device in FIG. 1;

FIG. 5 is a schematic view in longitudinal section of the device in FIG. 1 in which cell cultures and oxygenation paths of the cells under cultivation are indicated;

FIG. 6 is an exploded and perspective view of the device for three-dimensional cell-culture in FIG. 1;

FIG. 7 is a view from below of the device in FIG. 1;

FIG. 8 is a perspective view of an adapter intended to house a pair of devices for three-dimensional cell-culture according to the invention, in order to position them in an observation zone of an observation instrument;

FIG. 9 is an image obtained with a fluorescent microscope that allows to display Ewing sarcoma cells, genetically modified to express a red fluorescent protein (dsRED) and loaded onto the device;

FIG. 10 is an image obtained with a fluorescent microscope that allows to display pancreatic adenocarcinoma cells marked with the Calcein-AM green fluorescent dye (Invitrogen In correspondence with) and loaded onto the device;

FIG. 11 is a growth diagram of a tumor line of pancreatic ductal adenocarcinoma inside the device; growth is monitored through a RealTime-Glo luminometric assay (Promega Italia Srl) and allows to measure growth by evaluating the light emitted (RLU=relative light unit) that is directly proportional to the number of viable cells present; the RLUs are detected with a luminometer;

FIG. 12 is a growth diagram of a tumor line of breast carcinoma inside the device; growth is monitored through RealTime-Glo and allows to measure growth by evaluating the light emitted (RLU=relative light unit) that is directly proportional to the number of viable cells present; the RLUs are detected with a luminometer;

FIG. 13 is a histogram that allows to display the number of pancreatic ductal adenocarcinoma cells grown inside the device at 48, 72 and 96 hours; the number of cells is estimated according to the relative light units (RLUs) obtained by RealTime-Glo and detected with a luminometer; the RLUs are proportional to the number of viable cells;

FIG. 14 is a histogram that allows to display the number of breast cancer cells grown inside the device at 48, 72 and 96 hours; the number of cells is estimated based on the relative light units (RLUs) obtained by RealTime-Glo and measured on the luminometer; the RLUs are proportional to the number of viable cells;

FIG. 15 is a diagram representing the linearity curves obtained by loading increasing numbers of tumor cells into the device and measuring the relative light units (RLUs) at the luminometer after different incubation times (10, 20, 40 and 60 minutes) with the RealTime-Glo reagent; for each curve the trend line was calculated and the value R² of the curve was generated, which indicates a constant growth trend (high reliability of the trend line if R² approaches or is equal to 1), as expected when the luminometric reagent is able to support and detect increasing and even very high numbers of cells;

FIG. 16 is a histogram showing the growth of tumor cells inside the device at 72 hours, by measuring with the luminometer the relative light units (RLUs) obtained by adding to the culture the RealTime-Glo luminometric reagent;

FIG. 17 is a histogram showing the number of cells present inside the device after 72 hours of culture, estimated using the relative light units (RLUs) generated by the cells initially loaded (known number) and the RLUs generated by the cells after they have been grown for 72 hours, considering that the RLUs are proportional to the number of viable cells;

FIG. 18 is a histogram showing the evaluation of the efficacy of the soluble TNF-related antitumor biological agent apoptosis-inducing ligand (sTRAIL) added to the cells grown inside the device (time 0=start of treatment) in comparison with the cells that they have not been subjected to any kind of treatment (NT=not treated); the evaluation is carried out by adding the RealTime-Glo reagent and subsequent measurement with the luminometer of the relative light units (RLUs) that are proportional to the number of viable cells; the measurement of RLUs is carried out after 6 hours and 24 hours of treatment;

FIG. 19 is a histogram showing the number of tumor cells present inside the device in the absence of treatment (NT) or following treatment with the biological agent (sTRAIL) at time 0 (start of treatment) and after 6 hours and 24 hours of culture; the number of cells is estimated using the relative light units produced following the addition of the RealTime-Glo luminometric reagent and knowing the number of cells present at the beginning of the treatment (time 0);

FIG. 20 is a histogram showing the growth of luciferase positive tumor cells (that is, genetically modified to express the enzyme luciferase) inside the device after 72 hours of culture, by measuring with a luminometer the relative light units (RLUs) obtained by adding to the culture the luciferin substrate (Perkin Elmer In correspondence with);

FIG. 21 is a histogram showing the number of luciferase positive tumor cells (that is, genetically modified to express the luciferase enzyme) present inside the device after 72 hours of culture; the number of cells is estimated using the relative light units (RLUs) generated following the addition of the luciferin substrate from the initially loaded cells (known number) and the RLUs generated by the cells after they have been grown for 72 hours, considering that the RLUs are proportional to the number of viable cells and expressing luciferase;

FIG. 22 is a histogram showing the number of luciferase positive tumor cells (that is, genetically modified to express the enzyme luciferase) present inside the device in the absence of treatment (NT) or following treatment with the biological agent (sTRAIL) at time 0 (start of treatment) and after 24 hours of culture; the number of cells is estimated using the relative light units produced following the addition of the luciferin substrate and knowing the number of cells present at the beginning of the treatment (time 0);

FIG. 23 is a histogram showing the number of luciferase positive tumor cells present inside the device in the absence of treatment (NT) or following treatment with the biological agent (sTRAIL) at time 0 (start of treatment) and after 24 hours of culture; the number of cells is estimated using the relative light units produced following the addition of the luciferin substrate and knowing the number of cells present at the beginning of treatment (time 0);

FIG. 24 is a histogram showing the results in terms of cell viability obtained respectively using RealTime-Glo on tumor cells and the luciferin substrate on luciferase positive tumor cells, following treatment with sTRAIL; with the same treatment both methods are able to produce a comparable result;

FIG. 25 is a histogram showing the growth of breast carcinoma cells loaded on the device compared with the same cells loaded and treated after 24 hours from seeding with the NAB paclitaxel chemotherapy drug (PTX; Abraxane®, Celgene) at a concentration of 200 nM; the growth is monitored by adding the RealTime-Glo and detecting with the luminometer the relative light units (RLUs) that are proportional to the number of viable cells;

FIG. 26 is a diagram showing the recovery from the device of the three-dimensional matrix on which the cells have grown, by incision of the oxygenation membrane with a scalpel; the matrix containing the cells is included in a methacrylate-based resin, generating a chemically polymerized cube and incorporating the matrix with the cells; the resin cube is cut with a microtome and the sectioned part is then made to adhere to a microscope slide that can be colored with various histological or immune-enzymatic colorings and displayed under the microscope;

FIG. 27 is a microscopic image of a sectioned part made by cutting a methacrylate cube containing the three-dimensional matrix on which the cells were grown; the slide on which the slice was made to adhere is colored with hematoxylin and eosin and allows to observe the sagittal section of the three-dimensional matrix colonized by the cells;

FIG. 28 is an image under a fluorescence microscope showing the co-culture inside the device of genetically modified tumor cells to express a red fluorescent protein and stromal cells colored with a green fluorescent dye, for example Calcein-AM;

FIG. 29 is an image under a fluorescence microscope showing the co-culture inside the device of genetically modified tumor cells to express a red fluorescent protein and lymphocytes colored with a green fluorescent dye, for example Calcein-AM;

FIG. 30 is a diagram showing the possibility of dissociating a tumor biopsy or healthy tissue in order to isolate the cells and load them into the device to make them grow on the three-dimensional matrix and then display them and treat them with one or more active principles to predict, in a personalized patient-specific manner, the response to treatment in terms of efficacy (on tumor cells) or safety (on healthy cells).

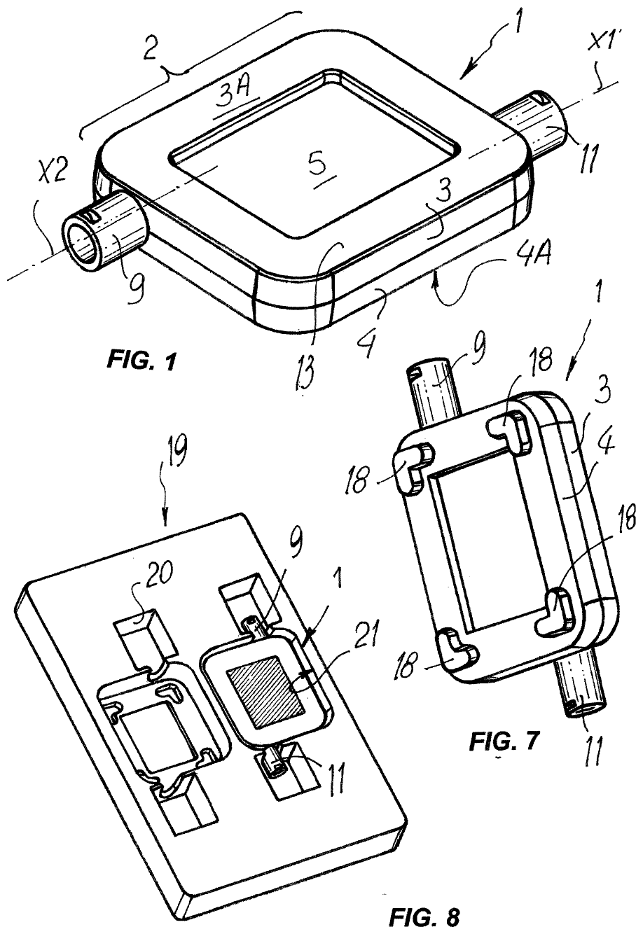

### DETAILED DESCRIPTION OF A PREFERRED EMBODIMENT

The culture device 1 comprises a box-shaped container body 2 which is formed by two equal and joined portions 3 and 4, which are substantially quadrangular in shape and which are preferably made of polymer material.

Each of the two portions 3 and 4 is provided with a perimeter frame zone 3A, 4A, which encloses inside it a membrane 5 and 6 of the gas permeable type, but impermeable to liquids.

The membranes 5 and 6 can be glued to the respective perimeter frames 3A, 4A, or, preferably, can be made simultaneously when the latter are made, during a molding step to make the two portions 3 and 4.

Between the two portions 3 and 4, when they are joined together, a three-dimensional matrix 7 is stretched and clamped, which is intended to receive and retain on itself a number of cells “C” to be cultivated.

The three-dimensional matrix 7 can be made of a known material such as NWF, which stands for Non-Woven Fabric.

The cells “C” are introduced into the container body 2 through a loading aperture 8 which is provided with a mouth 9 that extends toward the outside and which is associated with one of the two portions 3 or 4 while the other portion is associated with the other portion and is also provided with its own discharge aperture 10, provided with a mouth 11 which extends toward the outside like the mouth 9, but in the opposite direction with respect to it.

It should be noted that the two apertures 8 and 10 and the respective apertures 9 and 11 are offset from each other, even though they have parallel longitudinal axes “X1” and “X2”, to promote the expansion of a flow of the solution which carries in suspension the cells “C” to be cultivated that completely and homogeneously occupies the space delimited between the two portions 3 and 4 and defined as the culture chamber 12.

Through the mouth 9 a culture solution is introduced into the container body 2 in which the cells “C” are suspended, which are intended to be released on the three-dimensional matrix 7 so as to be cultivated, while instead, through the aperture 10 and the corresponding mouth 11, the transport solution is discharged after it has been deprived of the cells “C” transported.

The culture chamber 12 in the assembled configuration of the culture device 1 is divided into two semi-chambers by the three-dimensional matrix 7.

As can be seen in the drawings, each of the apertures 8 and 10 opens in correspondence with a respective semi-chamber of the culture chamber 2, in such a way that the flow of solution which carries in suspension the cells “C” to be cultivated follows in an obligatory manner a path that passes through the three-dimensional matrix 7, releasing them on the latter according to a distribution that is substantially homogeneous and expandable three-dimensionally.

In order to keep the two portions 3 and 4 in reciprocal contact when in the assembled configuration of the culture device 1 according to the invention, a perimeter edge 13 is provided which is applied by means of a molding step and which keeps the two portions 3 and 4 adherent to each other.

The device 1 is also provided, on at least one of the two portions 3 or 4, with a series of feet 18 to keep it slightly raised when it is resting on a surface. Advantageously, the polymer material used to make the edge 13 has a melting temperature lower than the melting temperature of the polymer material with which the two portions 3 and 4 are made: this is to allow, during the application step by means of hot pressing, to soften the edge 13 to complete its application and adhesion, without, however, reaching heating temperatures in the press that would soften the two portions 3 and 4 as well.

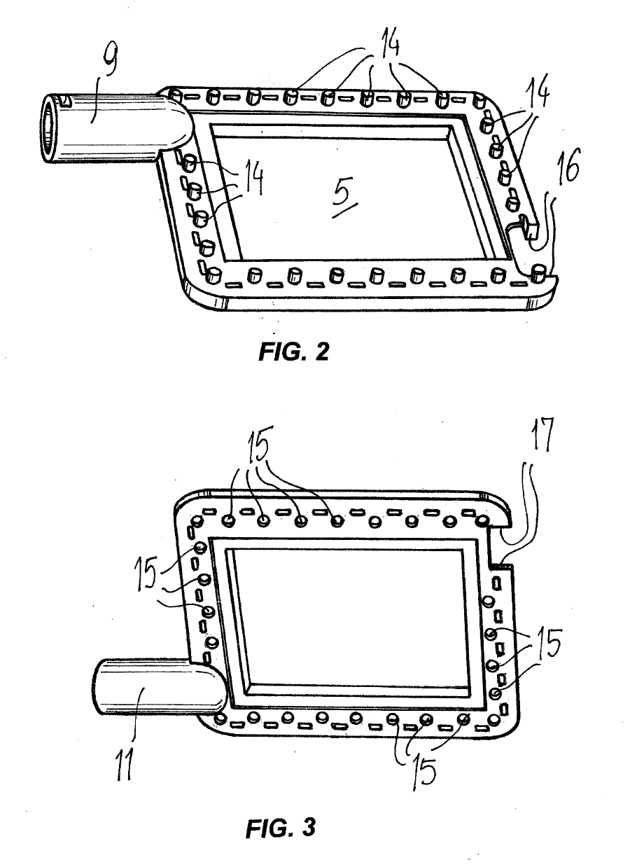

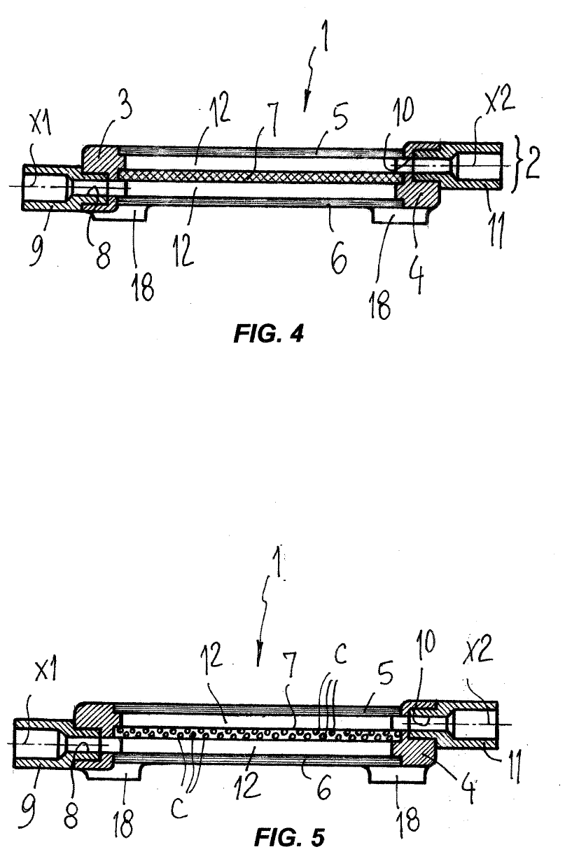

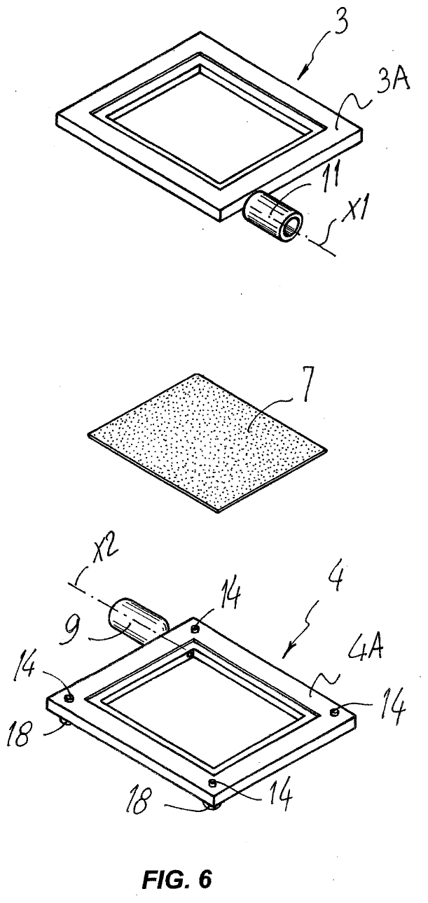

With reference to FIGS. 2 and 3, it can also be seen that the two portions 3 and 4 are provided, on the respective perimeter frame zones 3A and 4A, with a series of teeth 14 and corresponding holes 15 which are suitable to interlock with each other, keeping the coupling and alignment facing between the two portions 3A, 4A.

Moreover, in order to allow the coupling of the two portions 3 and 4, without the mouths 9 and 11 interfering with the respective perimeter frame zones 3A, 4A, respective hollows 16 and 17 are made in correspondence with the latter, one in each portion, to accommodate the corresponding mouths 9 and 11 in the assembled configuration of the device 1.

The latter can optionally be equipped with an adapter 19, as shown in FIG. 8, which, in the version shown by way of example, forms two concave seatings 20 and 21 shaped according to the outline of the device 1 and each of which is intended to receive a respective three-dimensional cell culture device 1.

The adapter 19 is used when it is necessary to put one or more culture devices 1 in an observation zone of a specific observation instrument, for example a microscope, or an acquisition instrument, for example a micro-plate reader, instruments not shown because they are known to the person of skill, to analyze the contents of the culture device 1.

In accordance with the culture method according to the invention, the culture device 1, if required, can be previously loaded with culture medium only, in order to wet the three-dimensional matrix 7, to facilitate the subsequent distribution of the cells “C” contained inside the cell suspension solution (so-called “priming”).

The cells are re-suspended in a culture medium to obtain a cell suspension which is loaded into the culture device 1 using a syringe.

The number of cells loaded is comprised in a range of from 30,000-1,000,000 for each cm² of the seeding surface of the culture device 1.

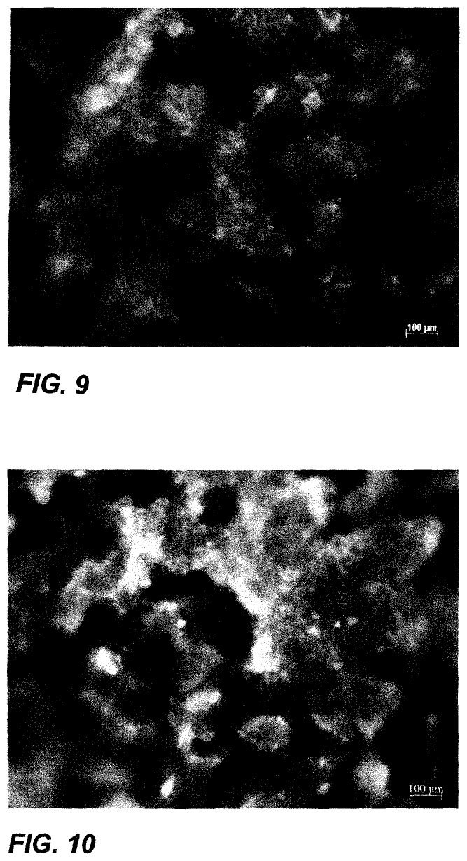

The cells “C” inside the latter can be observed through a fluorescence microscope, according to the following methods:
fluorescent cells “C”, that is, genetically modified to express a fluorescent protein included but not limited to GFP, YFP, CyanFP, DsRED; these cells can be directly observed inside the culture device 1 as can be seen in FIG. 9;
non-fluorescent cells “C”: these cells can be seen using cell tracers able to render the cells “C” fluorescent, as seen in FIG. 10.

In detail, the tracers are typically fluorescent probes or proteins that enter the cells “C”, after incubating them with a solution containing the selected tracer. The incubation is performed before the cells “C” are loaded into the culture device 1, or it is done directly inside it.

Tracers can be used in the solution to make cells “C” fluorescent for a short period (1-3 days), chosen, but not limited to Calceina-AM, CFSE, CMFDA green, orange CMRA, violet BMQC, CMTPX, Deep Red, or for a longer period (5-14 days), using the QTracker® cell labeling kit.

To make the cells “C” fluorescent, it is also possible to use fluorescent proteins that are made to enter the cells “C” through an incubation of 12-16 hours, and that make them fluorescent for up to 2 weeks (CellLight® Nucleus-GFP).

All the tracers cited above are distributed by the company Thermo Fisher Scientific.

Cell growth monitoring is performed using a luminometric reagent, for example RealTime-Glo.

The reagent contains a substrate that is metabolized by viable cells and an enzyme able to react with the metabolized substrate released by the cell; this reaction produces a light signal proportional to the number of viable and metabolically active cells; this signal is detected by a luminometer and expressed in relative light units (RLUs).

The reagent (consisting of two solutions, enzyme and substrate, which are mixed at the time of use) is added to the culture medium at a final concentration 1×, starting from a stock 1000×.

After an incubation ranging from 10 to 60 minutes, depending on the type of cells “C”, the light signal is detected through a common luminometer.

The light signal is directly proportional to the number of viable and metabolically active cells “C”.

In the three-dimensional system according to the invention, 30,000-1,000,000 per cm² of seeding surface of cells “C” are loaded for each culture device 1.

The RealTime-Glo reagent is added directly to the cell suspension and loaded together with the cells “C”, or is added at a later time together with fresh culture medium, after the cells “C” have been loaded into the culture device 1.

The detection of the light signal is performed in real time up to 96 hours after adding the reagent every 24 hours to the concentration 1× (from stock 1000×). Because the entity of the signal varies depending on the cell type and depends on the cell size, for some cell types a concentration of 2× (from stock 1000×) may be necessary at 96 hours, in order to guarantee a sufficient quantity of reagent also in the presence of very high cell densities.

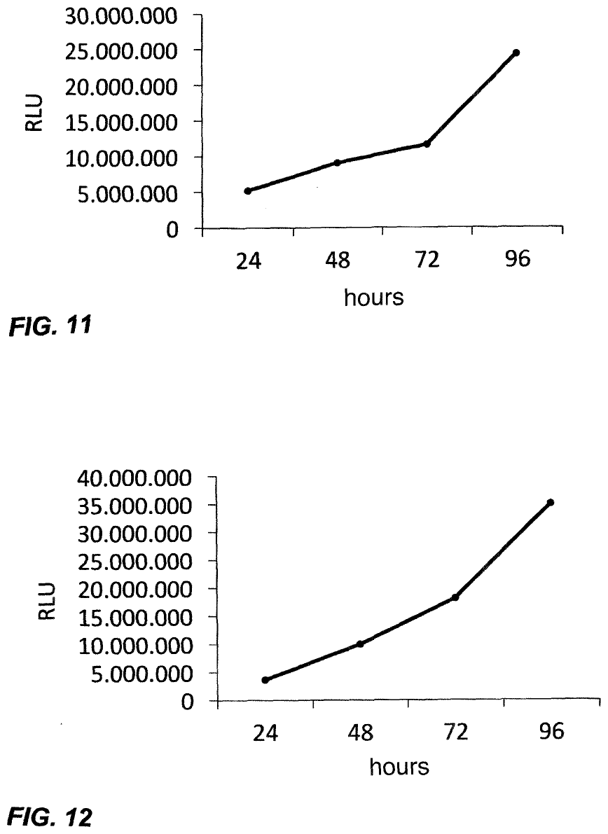

In the diagrams shown in FIGS. 11 and 12, the growth curve of a tumor line of pancreatic ductal adenocarcinoma (BxPC3) and of a breast tumor line (Bt549) are respectively shown.

The luminous intensity values acquired at the luminometer and expressed in “relative light units” (RLUs) are detected every 24 hours after adding culture medium to which the reagent RealTime-Glo 1× is added for the first 72 hours and 2× at 96 hours.

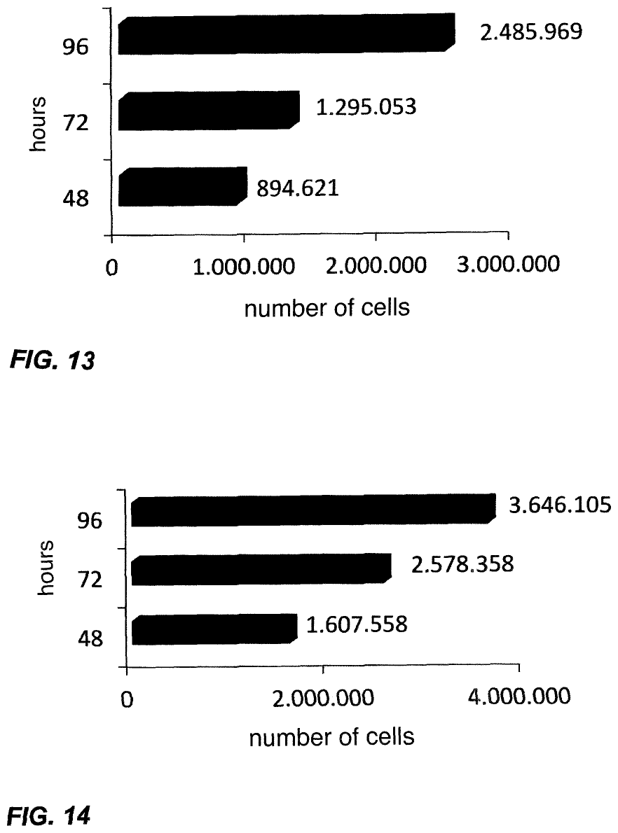

Since the RLU values are directly proportional to the number of viable cells “C”, knowing the number of cells “C” loaded initially (in this case approximately 560,000 cells for each culture device 1), it is possible to estimate the number of cells “C” grown inside the culture device 1, as can be seen in the diagrams shown in FIGS. 13 and 14 respectively for pancreatic ductal adenocarcinoma and breast cancer.

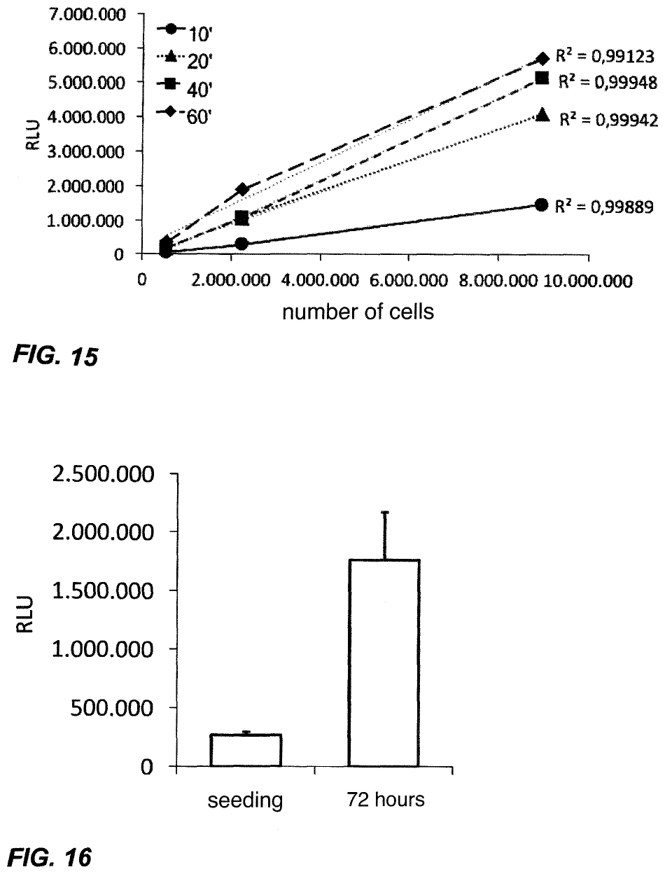

The diagrams shown in FIG. 15 show that it is possible to correlate the light signal with the number of viable cells “C” even in the presence of high cell densities, possibilities that are not usually envisaged by the protocol supplied with the reagent.

In the present invention, the linearity curve allows to establish whether there is a directly proportional correlation between the number of cells and the light signal; the curve is obtained by loading a progressive number of cells (in this case A673 cells, Ewing's Sarcoma line) inside the culture device 1 (560,000; 2,240,000; 8,960,000).

The trend line indicates that an element has an increasing or descending trend in a constant manner; a trend line is more reliable when the relative value of R squared (R²) is equal to or close to 1.

Monitoring the signal over time (detected at 10, 20, 40 and 60 minute intervals) allows to identify the stabilization of the signal, which may vary, however, depending on the cell type.

In the diagrams shown in FIG. 16, the culture device 1 was loaded with a Ewing Sarcoma tumor line and cell growth inside was monitored with the RealTime-Glo reagent until 72 hours.

The reagent is added to the cell suspension loaded in the culture device 1 and the light signal is acquired after 40 minutes of incubation and correlated with the number of cells “C” loaded (560,000 total cells).

The culture medium is changed with fresh medium every 24 hours.

After 72 hours of culture, the RealTime-Glo reagent is added to the medium and after a 40-minute incubation the light signal is acquired.

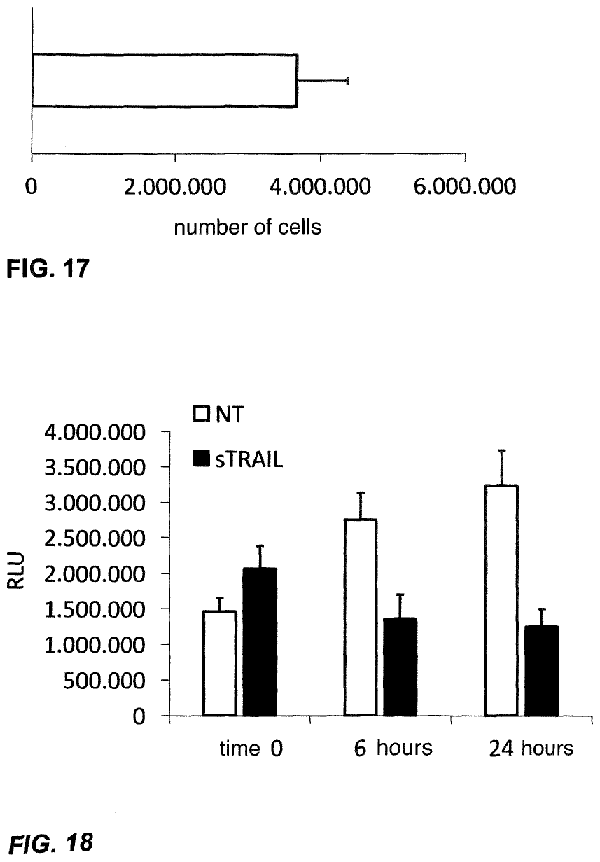

Typically, the light signal is directly proportional to the number of viable cells “C” and, therefore, it is also possible to estimate the number of cells “C” present inside the culture device 1, as can be seen in the diagram in FIG. 17.

After 72 hours of culture, the tumor cells “C” are treated with a biological cytotoxic agent (sTRAIL) which is added to the culture medium and loaded inside the culture device 1.

The efficacy of the agent is determined by measuring the light signal after 6 hours, without topping up the reagent, and 24 hours of treatment, topping up the reagent, as shown in the diagram in FIG. 18.

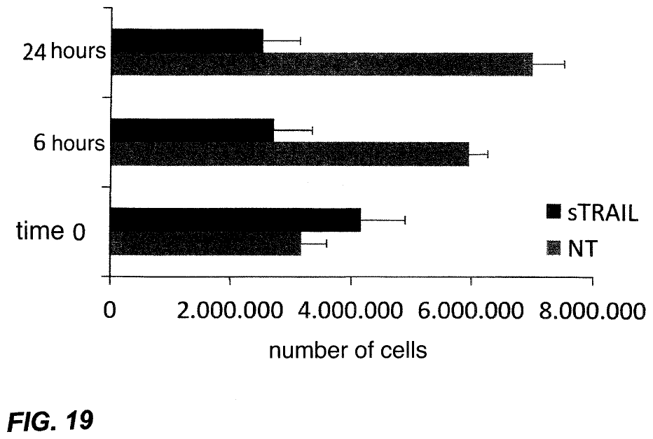

Using the RLUs and knowing the number of cells “C” loaded, it is possible to estimate the number of viable cells “C” present in the culture device 1 at various times after treatment, as shown in the diagram in FIG. 19.

Another method of monitoring growth inside the device provides to use genetically modified tumor cells “C” to express the luciferase enzyme.

In the presence of the luciferin substrate the cells “C” are able to metabolize the substrate, generating a light signal that is detected by means of a common luminometer.

This method is generally used in vivo: the formation of a tumor mass is induced in an experimental guinea pig by using human tumor cells “C” (xenotransplantation) that express luciferase.

After reaching a palpable tumor mass, whose formation requires 1-6 weeks depending on the type of tumor, we proceed with the anti-tumor treatment.

The efficacy of the treatment is evaluated by inoculating the luciferin substrate subcutaneously and monitoring the light signal with an in vivo “imaging” system that allows to localize and quantify the tumor mass, but which, however, does not allow to estimate the number of tumor cells “C”.

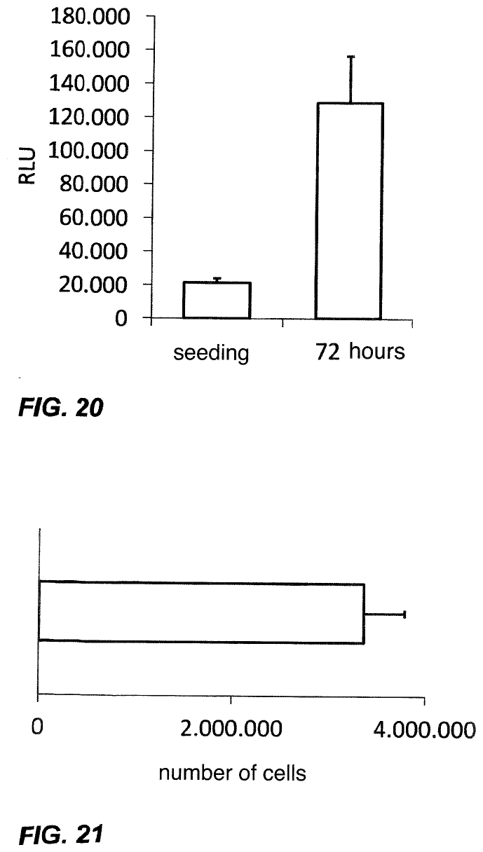

In the culture device 1 the tumor cells “C” which are luciferase-positive are grown for 72 hours and it is possible to monitor the extent of the growth and estimate the number of tumor cells “C” grown inside the culture device 1 by adding the luciferin substrate and detecting the light signal, as shown in the diagram in FIG. 20.

In FIGS. 20 and 21 it can be seen how the growth trend and the estimate of the number of tumor cells “C” present inside the device, evaluated at 72 hours, are in agreement with the data acquired with the different detection method based on the use of RealTime-Glo and shown in FIGS. 16 and 17.

The effectiveness of the sTRAIL biological agent, which is added to the culture medium and is loaded inside the culture device 1, is tested on the tumor mass inside the culture device 1 evaluated at 72 hours.

The efficacy of the treatment is determined after 24 hours by adding the luciferin substrate which is metabolized by the viable tumor cells “C” by the action of the luciferase.

Since the cells “C” that are dead or in apoptosis no longer produce the enzyme, they cannot metabolize the substrate and, consequently, they are no longer able to generate a light signal.

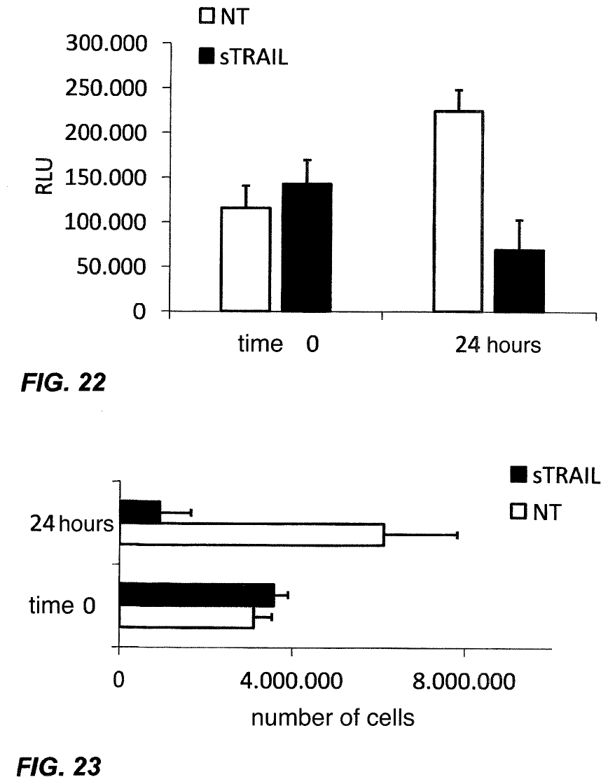

Therefore, the light signal is reduced in intensity as a consequence of the action of sTRAIL: the extent of the reduction allows to quantify the effectiveness of the treatment, as can be seen in the diagram in FIG. 22.

Using the RLUs and knowing the number of cells “C” loaded, it is possible to estimate the number of residual viable cells “C” present in the culture device 1 at various time intervals after treatment, as shown in FIG. 23.

By analyzing the RLUs obtained with both detection methods, the percentage of cell viability is calculated following treatment with the biological agent, putting the untreated control equal to 100% and obtaining the percentage of viability of the treated samples.

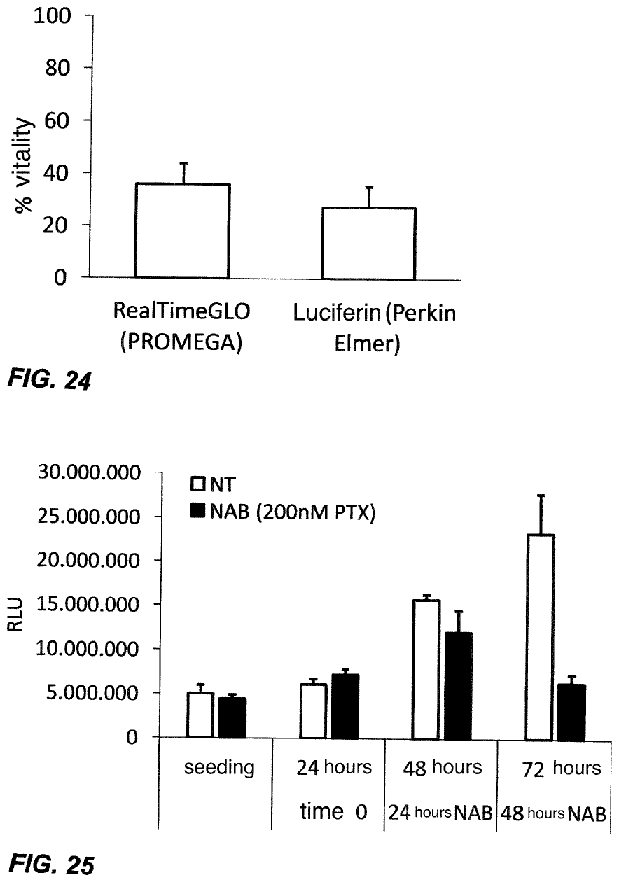

As can be seen in the diagram in FIG. 24 both detection methods generate a respective percentage of viability that is comparable with the other percentage.

The possibility of obtaining the same result with two different detection methods confirms a high degree of reliability of the method according to the invention and allows to underline its versatility, that is, the applicability of different systems for evaluating the pharmacological response.

The person of skill also knows that the monitoring of the growth of the cells “C” loaded and the quantification of the viable cells “C” present inside the device is also obtained using fluorimetric means.

Through the use of a common fluorimeter, the fluorescence emitted by cells “C” made fluorescent through gene modification or the use of fluorescent dyes, is quantified by generating a fluorescence intensity value (FI).

The FI value allows to estimate the number of residual viable cells “C” present in the culture device 1 at various time intervals after treatment.

Due to these characteristics and to the increased number of cells that constitute the three-dimensional tumor mass, the three-dimensional cell culture device and method according to the invention are located at an intermediate level between the miniaturized two-dimensional cell culture models, so-called in vitro, and in vivo cell culture models.

The invention allows to overcome the problems due to over-efficacy and low predictivity generated by miniaturized two-dimensional cell cultures, in which the three-dimensional structure of the in vivo cell is not respected and, on the contrary, a limited number of tumor cells “C” is associated which can be made to grow inside a multi-well plate.

In accordance with the invention, as well as molecular target drugs, the efficacy of conventional chemotherapeutic agents can be tested.

In the example shown in FIG. 25, mammary carcinoma cells (Bt549) are loaded into the culture device according to the invention and cultivated for 24 hours before being treated with NAB paclitaxel (PTX, trade name Abraxane®, produced by Celgene), a conventional chemotherapy drug also used for the treatment of breast carcinomas, which is added to the culture medium. Monitoring the growth with the RealTime-Glo reagent up to 72 hours of treatment allows to quantify the effectiveness of the chemotherapy drug that acts as a cytostat, firstly slowing down the growth of the tumor and finally inducing apoptosis.

To perform histological studies, the culture device 1 is incised in its central part with a scalpel, in order to recover the internal three-dimensional matrix 7 which houses the cell mass.

The three-dimensional matrix 7 is dehydrated by means of aqueous solutions at increasing concentrations of ethanol (alcoholic scale) up to 100% ethanol and subsequently included in methacrylate, as shown in the diagram in FIG. 26.

The matrix containing the cells is inserted into a liquid methacrylate solution; the solution is polymerized following the chemical reaction promoted by a catalyst, obtaining a solid block of methacrylate resin incorporating the matrix with the cells.

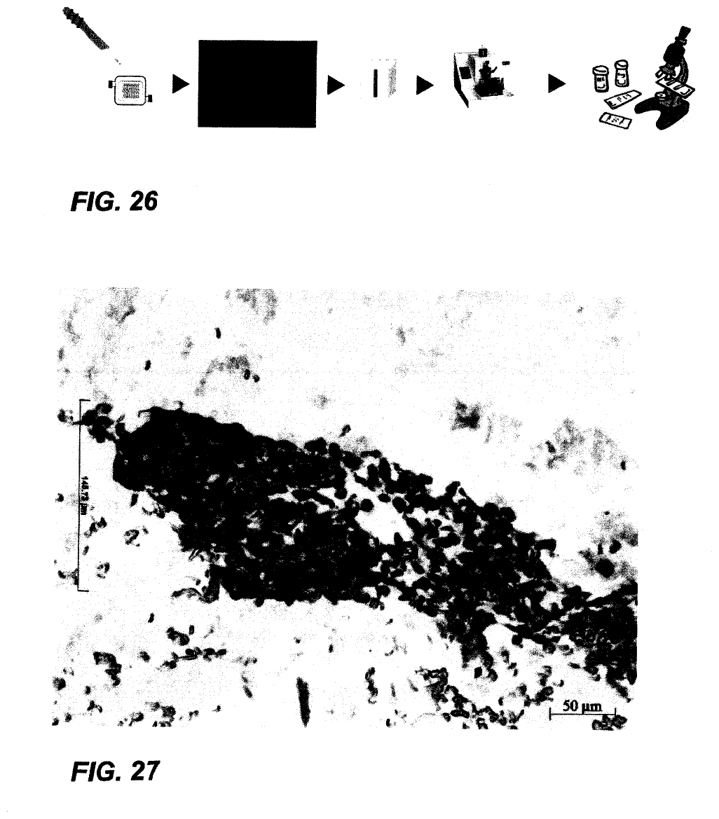

The block is then cut with a microtome in sagittal section and the slices obtained are made to adhere to a common microscope slide and can be colored to analyze the morphology of the tissue (hematoxylin and eosin) or used for immune-enzyme reactions intended to identify specific antigens (immune-histochemistry). FIG. 27 shows a hematoxylin eosin relating to sarcoma cells cultivated inside the culture device 1 according to the invention, subsequently included in methacrylate in order to obtain a sagittal section which allows to verify the colonization of the thickness of the matrix by the tumor cells.

Different cell types are also loaded inside the culture device 1, setting up a three-dimensional co-culture that can be used to:
evaluate the efficacy of the active principles and treatments on tumor cells even in the presence of components of the tumor microenvironment including but not limited to the extracellular matrix and stromal, hematopoietic and vascular elements in order to bring the complexity of the microenvironment closer to the in vivo situation with the aim of increasing the predictive response;
evaluate the effect of cellular effectors including, but not limited to, lymphocytes, CAR-T lymphocytes, mesenchymal cells, genetically modified mesenchymal cells on the target cells;
recreate complex organotypic cultures comprising different cell types.

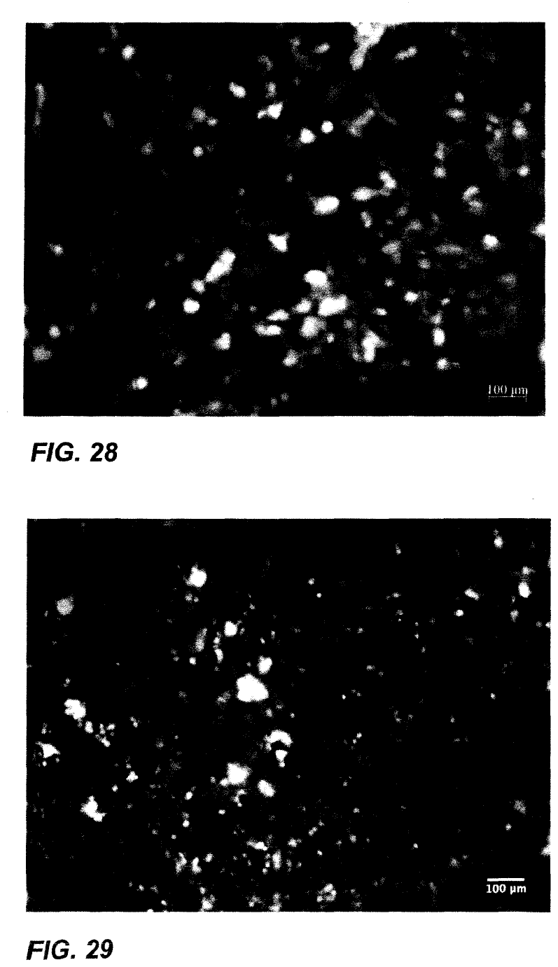

FIGS. 28 and 29 show some examples of co-culture cells inside the culture device 1 according to the invention.

The different cell types can be marked with fluorescent tracers of different colors in order to distinguish the different components.

Tumor cells “C” that are luciferase-positive can also be used, in order to determine the effect of the effector on the target through the use of the luciferin substrate, as previously mentioned.

The culture device 1 can be loaded with cells “C” of primary tumor isolated from a biopsy.

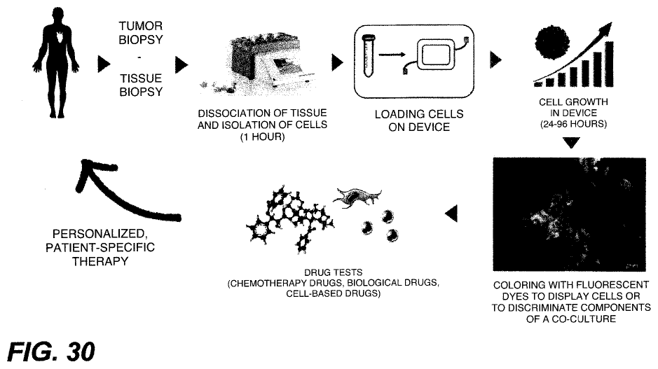

The biopsy is automatically dissociated and the isolated tumor cells “C” are loaded into the culture device 1, allowing to test active principles and laying the foundations for a process of personalized therapy, as indicated schematically in FIG. 30.

All the procedures described above can also be applied to healthy cells “C” including, but not limited to, cells from hepatic, splenic, pancreatic-biliary, cardiac, tracheo-broncho-pulmonary, epithelial-piliferous, gastro-intestinal, osteo-medullary, adipose, cartilaginous, central and peripheral nerve, oral-pharyngeal, thyroid, vascular, gonadal, uterine, cutaneous and subcutaneous tissue.

The cells “C” can be modified or not modified genetically to alternate their properties, as in the case of induced somatic progenitors (iPS) that are undifferentiated or differentiated into mesodermal, endodermal or ectodermal lines.

The cells “C” are grown in the culture device 1 to carry out cytotoxicity studies and to evaluate the possible side effects of active principles, treatments or biocompatibility studies.

The functioning of the culture device 1 according to the invention is as follows.

In a first operating step, the culture chamber 12 is primed with culture medium with which the three-dimensional matrix 7 is imbibed.

Subsequently, through the aperture 8, the solution carrying the cells “C” is introduced, which occupies the chamber 12, releasing the cells “C” on the surfaces of the three-dimensional matrix 7.

At the same time, the solution is discharged through the aperture 10.

The oxygenation of the cells “C” during the culture with oxygen coming from the outside occurs through the permeable membranes 5 and 6.

The cells “C” in culture are marked with fluorescent dyes and are displayed under a microscope with which their growth is also evaluated.

The efficacy or toxicity of an active principle to be tested is evaluated using viability assays of the cells “C”.

If required, the three-dimensional matrix 7 can be removed from the culture device 1 by cutting one of the membranes 5 or 6.

In practice it has been found that the invention obtains the intended purposes.

The invention as conceived is susceptible to modifications and variants, all of which come within the concept of the invention.

Furthermore, all the details can be replaced with other technically equivalent elements.

In practical implementation, the materials used as well as the shapes and sizes may be chosen as desired, according to requirements, without thereby abandoning the field of protection of the following claims.

1. Three-dimensional cell culture device comprising:
a container body of cells to be cultivated formed by a first semi-portion and a second semi-portion facing each other and attached together with attachment means;
a culture compartment which is defined between said first semi-portion and second semi-portion;
a three-dimensional substrate for the engraftment or support of cells to be cultivated which is located in said culture compartment; and
an inlet of a transport solution of cells to be cultivated, and an outlet that connects said culture compartment with the outside, to discharge said transport solution;
wherein at least one of said first semi-portion and second semi-portion comprises oxygenation means of said cells to be cultivated.

2. Device as in claim 1, wherein said oxygenation means comprise a first membrane associated with said first semi-portion and a second membrane associated with said second semi-portion, said first membrane and second membrane being permeable to gases and impermeable to liquids.

3. Device as in claim 2, wherein said first and second membranes are in one piece with the respective first and second semi-portions.

4. Device as in claim 1, wherein said attachment means comprise a perimeter edge which has a C-shaped cross-section and in whose cavity the perimeter edges of said first and second semi-portions are received.

5. Device as in claim 1, wherein interlocking elements are interposed between said first and second semi-portions, designed to keep them coupled together.

6. Device as in claim 1, wherein said three-dimensional substrate has edges held between said first semi-portion and second semi-portion and divides said culture compartment into a first semi-compartment and a second semi-compartment symmetrical with respect to each other.

7. Device as in claim 6, wherein said first semi-compartment and second semi-compartment are devoid of internal deviating or supporting elements.

8. Device as in claim 1, wherein said container body comprises a plurality of resting feet on a supporting surface.

9. Kit for three-dimensional cell culture, comprising a device for three-dimensional cell culture as in any claim hereinbefore and an adapter conforming at least one removable hollow housing seating of said culture device.

10. Method for three-dimensional cell culture comprising the following steps:
loading in a three-dimensional cell culture device a known number of cells to be cultivated, obtaining a known number of cells cultivated on a three-dimensional substrate contained in said three-dimensional cell culture device;
wherein before said loading, the method comprises:
carrying out a pre-filling of said three-dimensional cell culture device with a culture medium only;
and wherein after said loading, the method comprises:
monitoring cell viability at pre-established intervals of time;
detecting cell growth in said time intervals by said monitoring;
introducing at least one active principle to be tested into said cell culture device;
detecting with said monitoring a percentage of residual viable cells present in said three-dimensional cell culture device after introducing said at least one active principle to be tested;
deducing a rate of efficacy/toxicity of said at least one active principle to be tested as a ratio/proportion between said percentage of residual viable cells and said known number of cells.

11. Method as in claim 10, wherein said monitoring is carried out by selecting between luminometric or fluorimetric assays.

12. Method as in claim 11, wherein said luminometric assays are selected between luminometric assays applied to unmodified cells or luminometric assays based on cells genetically modified to express the luciferase gene.

13. Method as in claim 11, wherein said fluorimetric assays are selected from fluorimetric assays applied to fluorescent cells, genetically modified and designed to express a fluorescent protein, or fluorimetric assays applied to originally non-fluorescent cells made fluorescent with cell tracer means.

14. Method as in claim 10 wherein said active principle comprises cell-based agents.

15. Method as in claim 10, wherein said cells are human or animal cells.

16. Method as in claim 10, wherein said cells are healthy or tumor cells.

17. Method as in claim 10, wherein said healthy cells are pan-tissue-derived cells including genetically modified cells.

18. Device as in claim 2, wherein interlocking elements are interposed between said first and second semi-portions, designed to keep them coupled together.

19. Device as in claim 3, wherein interlocking elements are interposed between said first and second semi-portions, designed to keep them coupled together.

20. Device as in claim 4, wherein interlocking elements are interposed between said first and second semi-portions, designed to keep them coupled together.
